(19) United States
(12) Patent Application Publication
(10) Pub. No.: US 2013/0197369 A1
(43) Pub. Date: Aug. 1, 2013

(54) Remote health monitoring and tracking system

(71) Applicant: Jiannan Xiang, Suwanee, GA (US)

(72) Inventor: Jiannan Xiang, Suwanee, GA (US)

(21) Appl. No.: 13/385,025

(22) Filed: Jan. 30, 2012

(51) Int. Cl. A61B 5/0255; A61B 6/00; A61B 5/021; A61B 7/00; A61B 5/00; A61B 5/01

(52) U.S. Cl. 600/474; 600/300; 600/500; 600/549; 600/485; 600/528

(57) ABSTRACT

This utility model patent makes public a remote health monitoring and tracking system. The system includes the product and the service end. The product includes wireless communication module(1), physiology sensor(2), physiological signal processing circuit(3) and microprocessor(4). Among these, (2) connects (3), (3) connects (4), and (4) connects (1). The service end includes server that can connect to Internet. The database, map software and physiological data analysis software are installed on the server. The wireless communication module, with integrated GPSone technology and embedded TCP/IP, connects to the server through TCP/IP. Compared with the current technologies, this utility model is easier to use, and can monitor and track the health conditions of users remotely. Any abnormal situation happens to the users, their families and emergency center will be noted. Besides, it has GPSone function; the stored physiological data can be used as reference for medical professionals.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The aging of population becomes more and more serious, and the health care of the old therefore becomes a top social problem. The traditional health monitoring instruments of body temperature, pulse, blood pressure etc. can be only used to measure, record and present the physiological data, but can't remotely monitor and track the health conditions of the users through wireless communication. And what's worse, they are inconvenient to carry. If unpredictable situations happen to the users, they may lose the best possible care and first aid due to untimely notifying their families and emergency center.

The prior and related art named as A Medical System with Functions of Remote Monitoring, Detection and Alarm, make public a system of detecting and caring human body's temperature, sweatiness degree, pulse and blood pressure. After detecting abnormal data on temperature, sweatiness degree, pulse and blood pressure, this system of wrist monitoring device, connects to the mobile phone and sends signal for help to communication center through its inner circuit. The device is also installed GPS module for positioning. Although the system can give an alarm for help through communication mode, it needs the users to have a mobile phone which has to connect to circuit. Therefore, it's inconvenient to use and carry. And the PGS function is not available when users are indoors or the signal is blocked by buildings. In this case, the users can not be found timely in emergency, which will cause potential safety hazard.

### BRIEF SUMMARY OF THE INVENTION

In allusion to the deficiency of existing technologies, this utility model device is a convenient remote health monitoring and tracking system, aiming to transmit the physiological data of the user collected by the product to the service end through TCP/IP for further data processing and analysis. It also has the functions of alarming and positioning. Any abnormal situation happens to the users, their families and emergency center will be noted. Besides, it has GPSone function; the stored physiological data can be used as reference for medical professionals.

### BRIEF DESCRIPTION OF THE SEVERAL VIEWS OF THE DRAWING

Having thus described the invention in general terms, reference will now be made to the accompanying drawings, which are not necessarily drawn to scale, and wherein:

FIG. 1: Structural Representation of the Remote Health Monitoring and Tracking System

FIG. 2: Structural Representation of Exploit Example One

FIG. 3: Structural Representation of Exploit Example Two

FIG. 4: Structural Representation Between Pulse Measurement Device and Bluetooth Chip in Exploit Example Two

FIG. 5: Structural Representation Between Blood Pressure Measurement Device and Bluetooth Chip in Exploit Example Two

FIG. 6: Structural Representation Between Body Temperature Measurement Device and Bluetooth Chip in Exploit Example Two

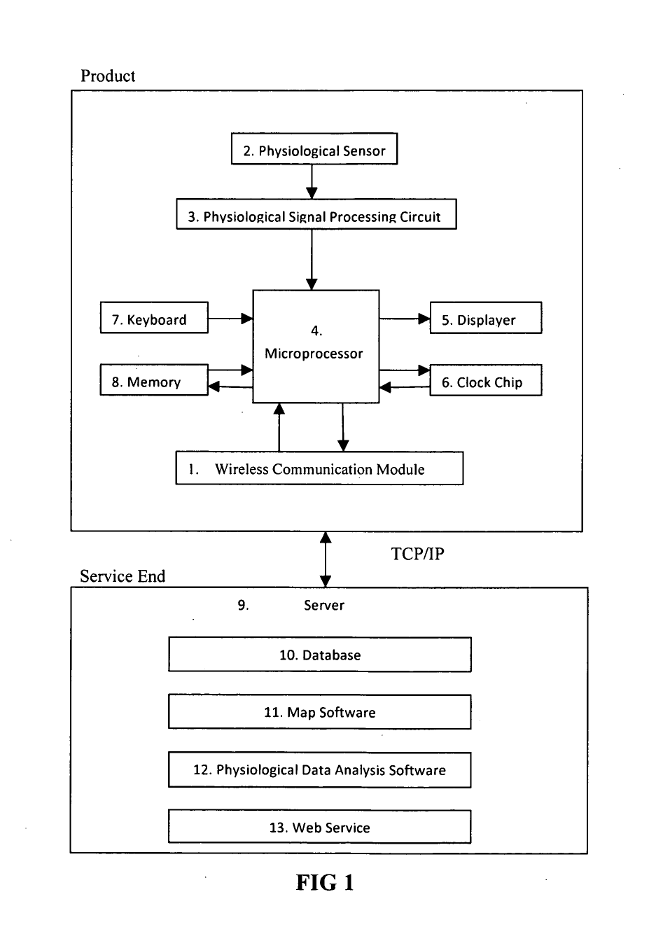

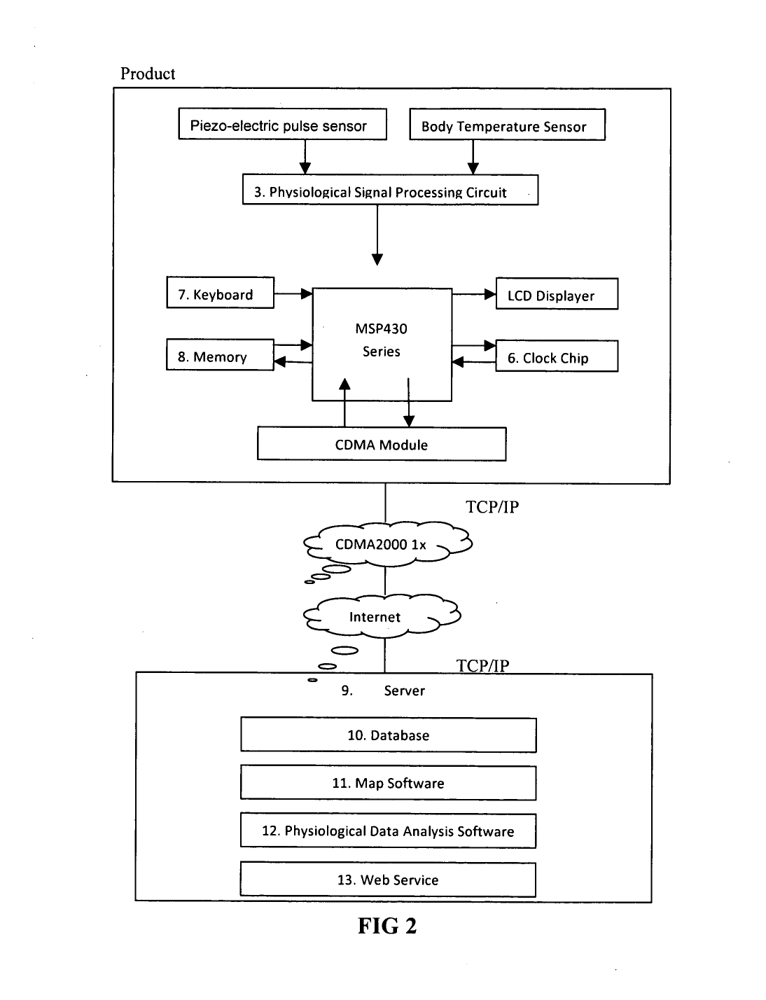

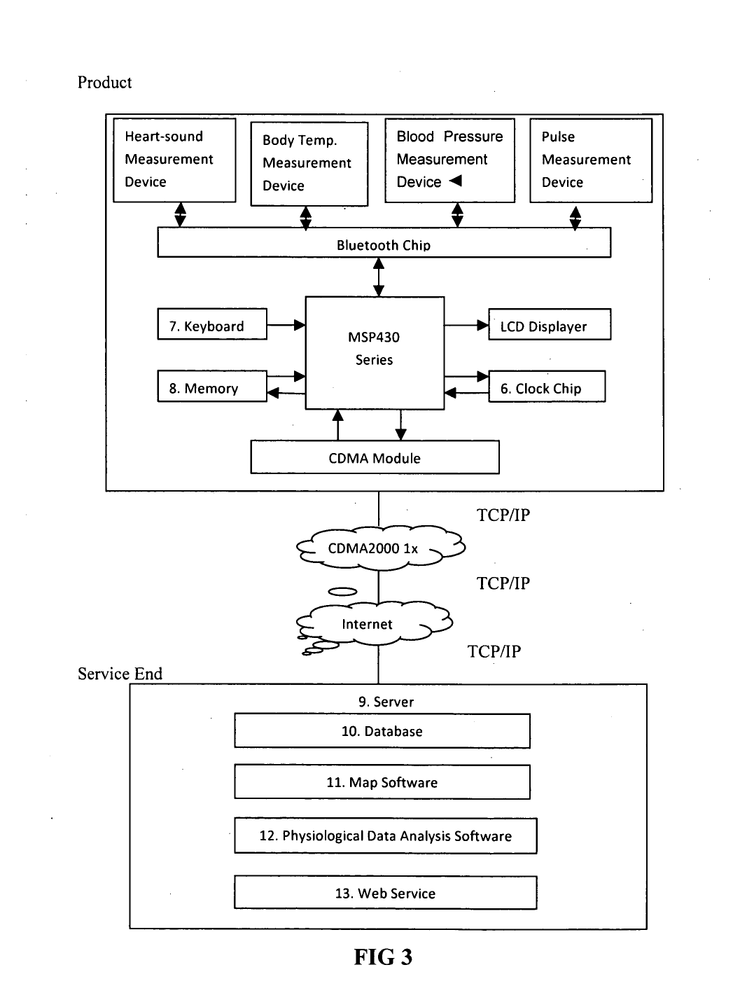

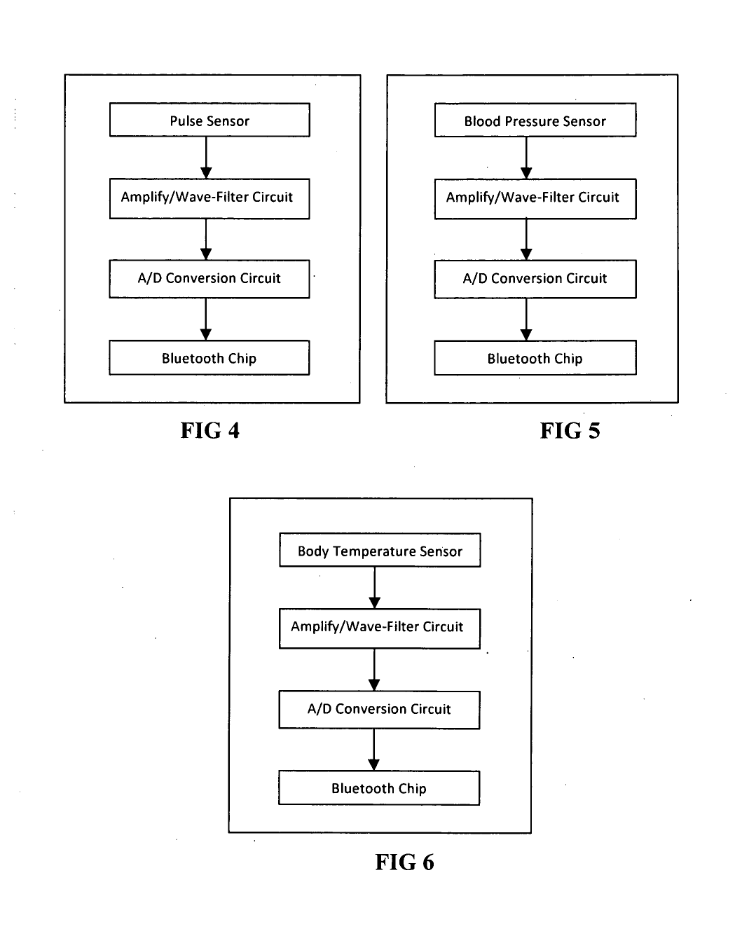

### DETAILED DESCRIPTION OF THE INVENTION

This utility model adopts the following technical schemes: This utility model patent makes public a remote health monitoring and tracking system. The system includes the product and the service end. The product includes wireless communication module, physiology sensor, physiological signal processing circuit and microprocessor. The service end includes server that can connect to Internet. The database, map software and physiological data analysis software are installed on the server. The wireless communication module, with integrated GPSone technology and embedded TCP/IP, connects to the server through TCP/IP. The mentioned product also includes displayer, clock chip, keyboard and memory, and they all connect to the microprocessor. The mentioned service end contains Web service. There is a Bluetooth chip between the mentioned physiological signal processing circuit and the mentioned microprocessor; physiological signal processing circuit connects the Bluetooth chip through Bluetooth protocol; and the Bluetooth chip connects microprocessor.

### I. Wireless Communication Module

It's integrated with GPSone technology and embedded with TCP/IP to achieve the functions of positioning, data transmitting and voice call, etc. Receiving from and sending data to the server on service end is the important module to realize remote monitoring and positioning.

### II. Physiology Sensor

It collects physiological data of the users' pulse, blood pressure, body temperature, etc. Physiology sensor can be a pulse sensor, body temperature sensor, blood pressure sensor or heart-sound senor. The above-mentioned pulse sensor can be a photoelectric sensor or piezo-electric sensor. It can calculate the characteristic quantity based on the detected pulse wave. Then blood pressure is measured based on the characteristic quantity. The above-mentioned body temperature sensor can be a infrared temperature sensor. It can measure body temperature through thermal release from the wrist.

### III. Physiological Signal Processing Circuit

It amplifies and filters waves of physiological data collected by physiology sensor, as all the collected data are micro-electric signals and liable to be interrupted, they need to be amplified and wave filtered. After that, the circuit processes A/D conversion to convert electrical signals into digital signals.

### IV. Microprocessor

It mainly controls the collection and simple calculation of physiological data, data displayer, clock chip, wireless communication module, and response to an interruption of keyboard. Microprocessor can be high-powered MSP430 series, single chip of 51 series, or embedded DSP or ARM processor.

### V. Displayer

It's used to display the remaining charge, pulse frequency, blood pressure, body temperature, clock time, signal intensity connecting with the service end, etc. If the microprocessor doesn't have the LCD driving function, a driving circuit needs to be equipped.

### VI. Clock Chip

It controls input and output of BCD code of the year, month, week, date, hour, minute and second.

### VII. Keyboard

It consists of the product's start button, stop button, reset button, clock adjustment button and emergency alarm button, etc.

### VIII. Memory

It's used to temporarily store the collected data and location parameter.

### IX. Server

The server, together with wireless communication module, bring about the connection of TCP/IP, sending and receiving data (including physiological parameter and location parameter), and storing data into the database. Servers from different companies such as IBM can be used.

### X. Database

It's used to store and keep data of every product, including physiological parameter and location parameter.

### XI. Map Software

It's used to display the received location parameter (longitude, latitude, speed) from the product on the map, through which, the users can be found by their families and emergency center quickly and accurately to receive timely care.

### XII. Physiological Data Analysis Software

It's used to analyze the received physiological data from the product. Any abnormal situation happens to the users, their families and emergency center will be noted.

### XIII. Web Service

It provides service of reviewing history data, including change curve of pulse wave, blood pressure and locations, etc. The medical professions and the families can refer to the history data through the users' ID and passwords or mobile phones or computers.

In order to make it easy to use, convenient to carry and real-time detect automatically, the above-mentioned product can be a watch-style device. The product is slightly larger than a normal watch. When the users wear it, it can collect physiological data automatically. The described physiology sensor can be fixed on the inner side of the watchband. The watchband is made of shrinkable material, so as to aim the sensor accurately at the artery. The specifications of watchband can be made into different kinds to fit different sizes of users' wrist. If the Bluetooth chip is fixed between physiological signal processing circuit and microprocessor, the photoelectric sensor can be chosen as pulse sensor, and made into fingertip style, so the users can wear the sensor on tip of finger to measure pulse; body temperature sensor can be made as a plaster device to stick under the arm to measure temperature; heart-sound senor can be placed on vest to measure heart-sound. Then all these pulse data, body temperature data and heart-sound data will be transferred into the Bluetooth chip of the product through Bluetooth protocol.

### Detailed Exploit Examples

The optimized exploit examples of this utility model will be further detailed as follows:

### Exploit Example One

A remote health monitoring and tracking system, including product and server end. The product is watch-style and can be worn on the wrist. The product includes wireless communication module1, physiology sensor2, physiological signal processing circuit that can amplify/filter and achieve A/D conversion3 and microprocessor4, displayer5, clock chip6, keyboard7 and memory8; physiology sensor2 connects physiological signal processing circuit3; physiological signal processing circuit3 connects microprocessor4; microprocessor4 connects wireless communication module1, displayer5, clock chip6, keyboard7 and memory8 separately. The physiology sensor2 is fixed on the inner side of watchband. The watchband is made of shrinkable material. The service end includes server that can connect to Internet9. The database10, map software11 and physiological data analysis software12 and Web service13 are installed on the server9.

The mentioned wireless communication module1 is integrated with GPSone technology and embedded with TCP/IP and CDMA module (ANYDATA DTGS-800). It connects to server9 through TCP/IP. The microprocessor4 is a single chip of MSP430 series, connects wireless communication modulel by serial port and controls by AT commands. The physiological sensor2 includes piezo-electric pulse sensor and infrared thermopile temperature sensor with a model number of ZTP135S-R published by General Electric Company (GE). The server9 can calculate the characteristic quantity based on the detected pulse wave. Then blood pressure is measured based on the characteristic quantity. The server9 can also realize TCP/IP connection with wireless communication module1 of several products. The displayer5 adopts WGM12864M. The clock chip6 adopts S-3530. The keyboard7 consists of the product's start button, stop button, reset button, clock adjustment button and emergency alarm button.

### Exploit Example Two

The parts same as in Exploit Example One will not be repeated. The differences lie in: The wireless communication modulel adopts FIDELIX FD800. The physiological sensor2 includes photoelectric pulse sensor, body temperature sensor, blood pressure sensor or heart-sound senor, not measure blood pressure by characteristic quantity of pulse waves. The physiological signal processing circuit3 includes amplify/wave-filter circuit and A/D conversion circuit. There is a Bluetooth chip between physiological signal processing circuit3 and microprocessor4; physiological signal processing circuit3 connects the Bluetooth chip through Bluetooth protocol; and the Bluetooth chip connects microprocessor4. The photoelectric pulse sensor is made into fingertip style, and worn on tip of finger to measure pulse; body temperature sensor is made into a plaster device to stick under the arm to measure temperature; heart-sound senor is placed on vest to measure heart-sound. Then all these pulse data, body temperature data and heart-sound data will be first transferred into the Bluetooth chip of the product through Bluetooth protocol, and then transferred into the microprocessor4.

### Exploit Example Three

The parts same as in Exploit Example One will not be repeated. The differences lie in: The microprocessor4 is DSP embedded processor.

What I claim as my invention is:

1. A remote health monitoring and tracking system, including product and server end. The system is characterized by: The product includes wireless communication module(1), physiology sensor(2), physiological signal processing circuit that can amplify/filter and achieve A/D conversion(3) and microprocessor(4). Among these, physiology sensor(2) connects physiological signal processing circuit(3), physiological signal processing circuit(3) connects microprocessor(4), and microprocessor(4) connects wireless communication module(1). The service end includes server that can connect to Internet(9). The database(10), map software(11) and physiological data analysis software(12) are installed on the server(9). The wireless communication module(1), with integrated GPSone technology and embedded TCP/IP, connects to the server(9) through TCP/IP.

2. According to the claim 1, the system is characterized by: The product also includes displayer(5), clock chip(6), keyboard(7) and memory(8), and they all connect to the microprocessor(4).

3. According to the claim 1 or 2, the system is characterized by: The server(9) contains Web service(13).

4. According to the claim 1 or 2, the system is characterized by: There is a Bluetooth chip between physiological signal processing circuit(3) and microprocessor(4); physiological signal processing circuit(3) connects the Bluetooth chip through Bluetooth protocol; and the Bluetooth chip connects microprocessor(4).

5. According to the claim 1 or 2, the system is characterized by: the above-described product is watch-style.

6. According to claim 5, the system is characterized by: The physiology sensor(2) is fixed on the inner side of watchband. The watchband is made of shrinkable material.

7. According to the claim 1 or 2, the system is characterized by: The microprocessor(4) is either a single chip or embedded processor.

8. According to the claim 2, the system is characterized by: The keyboard(7) consists of the product's start button, stop button, reset button, clock adjustment button and emergency alarm button.

9. According to the claim 1 or 2, the system is characterized by: physiology sensor(2) is a pulse sensor, body temperature sensor, blood pressure sensor or heart-sound senor.

10. According to the claim 9, the system is characterized by: The above-mentioned pulse sensor is photoelectric sensor or piezo-electric sensor; the above-mentioned body temperature sensor is infrared temperature sensor.
